(19) United States
(12) Patent Application Publication
(10) Pub. No.: US 2009/0037116 A1
(43) Pub. Date: Feb. 5, 2009

(54) SYSTEMS, DEVICES, AND METHODS FOR ANALYZING MACROMOLECULES, BIOMOLECULES, AND THE LIKE

(75) Inventor: Barry Patrick Benight, San Jose, CA (US)

Correspondence Address: SEED INTELLECTUAL PROPERTY LAW GROUP PLLC, 701 FIFTH AVE, SUITE 5400, SEATTLE, WA, 98104, US

(73) Assignee: PORTLAND BIOSCIENCE, INC., Portland, OR (US)

(21) Appl. No.: 11/971,770

(22) Filed: Jan. 9, 2008

Related U.S. Application Data
(60) Provisional application No. 60/884,161, filed on Jan. 9, 2007.
(60) Provisional application No. 60/947,597, filed on Jul. 2, 2007.

(51) Int. Cl. G01N 33/48

(52) U.S. Cl. 702/19

(57) ABSTRACT

Systems, devices, and methods for analyzing hybridization of target molecules to probes on substrate-bound oligonucleotide, peptide, or protein arrays. In one aspect, the system includes a computer-readable memory medium and a controller. The system may further include a computer-readable memory medium including thermodynamic data configured as a data structure for use in analyzing biological samples. In some embodiments, the data structure comprises a thermodynamic data section having: thermodynamic data representative of dangling ends of two or more bases; thermodynamic data representative of unpaired single strands of two or more bases adjacent to a Watson-Crick base pairing; thermodynamic data representative of unpaired single strands of one or more bases adjacent to a non-Watson-Crick base pairing; thermodynamic data representative of tandem base pair mismatches of two or more bases; thermodynamic data representative of length-dependent terminal mismatches of nucleic acid bases; thermodynamic data representative of terminal base pair mismatches, or combinations thereof.

### CROSS-REFERENCES TO RELATED APPLICATIONS

This application claims the benefit under 35 U.S.C. § 119(e) of U.S. Provisional Patent Application No. 60/884,161 filed Jan. 9, 2007 and U.S. Provisional Patent Application No. 60/947,597 filed Jul. 2, 2007.

### BACKGROUND

1. Technical Field

This disclosure generally relates to the fields of molecular biology, microbiology, bioinformatics, and biophysics and, more particularly, to systems, devices, and methods for analyzing hybridization of target molecules to probes on substrate-bound oligonucleotide, peptide, or protein arrays.

2. Description of the Related Art

Nucleic acid diagnostic testing has become a major focus for the fields of genomics, pharmacogenomics, proteomics, and genetic medicine just to name a few. Assay platforms capable of detecting the presence of genes, differential gene expression levels, and genetic variations constitute active areas of development. For example, deoxyribonucleic acid (DNA) arrays can simultaneously analyze the expression of hundreds of genes and permit systematic approaches to biological discovery.

DNA sequences in solution or in a semi-constrained solution (such as a micro-array) form duplexes with other available sequences based on, for example, the properties of the individual duplexes, the temperature of the solution, the relative concentrations of the DNA sequences, and the presence of other factors (e.g., salt concentration). Much of the computational research surrounding DNA is involved with finding similarities between sequences, especially in the face of mismatches, and insertions and deletions of one or more bases. Nearly all computational genetic approaches in the existing state of the art, however, treat the text-based identity of the bases making up the sequences as the only information necessary to determine the level of match or mismatch.

Nucleic acid diagnostic tests often employ strategies based on the hybridization principles of genetic material to DNA or RNA probes. These probes are generally designed in silico with the intent that they bind specifically with their perfectly matched targets. In practice, however, probes often bind to target sequences that are similar to their corresponding complementary target sequences. This cross-hybridization effect often skews the observed data from the expected data by signaling the presence of multiple sequences other than the expected target sequence. Cross-hybridization further complicates the data analysis by presenting numerous statistical problems, including the normalization of the data. Accordingly, there is a need to minimize cross-hybridization effects, as well as a need to better quantify cross-hybridization effects.

Often the sequence of nucleotides in DNA, or the sequence of amino acids in a protein or peptide, is represented as text strings indicative of the nucleotides or amino acids making up the sequence. For example, the sequence of nucleotides in DNA is often represented as a text string based on a four-letter alphabet (A, C, G, T) that symbolically codes for the corresponding nucleotide (e.g., adenine, cytosine, thymine and guanine). Accordingly, much of the sequence analysis, such as homology and similarity searches, protein functional analysis, motif searches, protein structure analysis, and the like often involve text-based search technologies and algorithms, as well as sequence alignment representations that compare the text of a sequence of interest to the text of other sequences.

In sequence alignment representations, sequences are written in rows arranged so that aligned residues appear in successive columns. Many of the available design routines rely on text similarity alignment routines to find, or generate and filter candidate probe sets. One problem with text-based search technologies and algorithms is that they fail to account for many of the secondary and tertiary structure effects associated with many macromolecules (e.g., nucleic acids, proteins, genomes, and the like). Another problem with text-based technologies and algorithms is that they take far too long to reliably compare a probe to a long genomic sequence.

A number of routines have been written to speed up text-based search algorithms. For example, most commonly used search queries employ the Basic Local Alignment Search Tool (BLAST) that looks for sequence homologies between a query sequence and selected genome sequences. Alignments are approximated by a search algorithm fashioned after the “seed” and “expand” Smith-Waterman method that identifies regions of local sequence text similarity and reports the likelihood that the match is the result of random chance.

BLAST has found primary utility in text-based recognition of patterns of sequence similarity used as indicators of evolutionary connectivity. BLAST is also commonly employed to deduce likelihood of duplex formation based on relative sequence homologies between probes and targets determined in text-based searches. But, as previously noted, text-based search technologies and algorithms like BLAST fail to account for some of the duplex interactions formed by probes and targets.

Another approach to speed up text-based search algorithms employs field programmable gate arrays (FPGAs) that distribute text-based comparison algorithms across hundreds or thousands of discrete processing elements for rapid parallel execution of text-based searches. But the FPGAs are designed to perform text-based searching and are therefore limited by the same problems that ultimately limit BLAST.

TIMELOGIC® biocomputing solutions has developed the DECYPHERBLAST™, a search engine using FPGA technology that parallelizes the BLAST search algorithm and has demonstrated improvements in both speed and performance at reduced costs. A shortcoming of this approach, however, is that genomic sequence searches are implemented using text-based approaches. Accordingly, probes designed using this search engine still suffer from cross-hybridization problems due to sequence interactions with other sequences, having dissimilar, non-homologous motifs, which are often unaccounted for in text-based technologies and algorithms approaches.

The present disclosure is directed to overcoming one or more of the shortcomings set forth above, and providing further related advantages.

### BRIEF SUMMARY

The letter code or text representation of DNA sequence (e.g., A, T, G, C) is one of the most basic representations and contains important information regarding the protein sequences encoded by DNA (e.g., codons). Unfortunately, the text representation of DNA does not provide much insight regarding the distribution of thermodynamic stability encoded in a DNA sequence. For example, influence of “non-natural” configurations such as mismatch hybrids containing tandem mismatches or misalignments between two strands results in contributions that are lost in text-based homology searches, but that might have an important influence on actual results (generation of cross-hybridization and false positives). Furthermore, sequence dependent thermodynamic stability may encode for physical, chemical, and functional characteristics of duplex DNA that is often unaccounted for in text-based homology searches. Approaches that account for and/or quantify, for example, cross-hybridization effects or the influence of “non-natural” configurations using thermodynamics may be better predictors of true behavior, than those approaches relying on text representations of DNA.

In one aspect, the present disclosure is directed to a data processing system for analyzing a biological sample. The system includes a computer-readable memory medium and a controller.

The computer-readable memory medium comprises thermodynamic data configured as a data structure for use in analyzing biological samples. In some embodiments, the data structure comprises a thermodynamic data section having: thermodynamic data representative of dangling ends of two or more bases; thermodynamic data representative of unpaired single strands of two or more bases adjacent to a Watson-Crick base (w/c) pairing; thermodynamic data representative of unpaired single strands of one or more bases adjacent to a non-Watson-Crick base pairing; thermodynamic data representative of tandem base pair mismatches of two or more bases; thermodynamic data representative of length-dependent terminal mismatches of nucleic acid base; thermodynamic data representative of terminal base pair mismatches, or combinations thereof.

In some embodiments, the controller is configured to compare an input associated with the biological sample to the thermodynamic data, and to generate a response based on the comparison. In some embodiments, the input associated with the biological sample comprises at least one of an output generated from a detected image of the biological sample applied to an array, gene expression data, nucleic acid sequence data, an n-dimensional expression profile vector of the biological sample, a genome of an organism, or combinations thereof.

In another aspect, the present disclosure is directed to a method in a computer system for analyzing nucleic acid probes. The method includes determining a first free energy value indicative of a duplex of a first nucleic acid probe and a first target nucleic acid sequence. The method may include determining a first minimum free energy value indicative of a lowest free energy value associated with a formation of each of one or more duplexes formed by the first nucleic acid probe and at least a second target nucleic acid sequence.

The method may further include determining a second minimum free energy value indicative of a lowest free energy value associated with the formation of each of one or more duplexes formed by the first nucleic acid probe and at least a second nucleic acid probe. The method may further include determining a difference between the determined first free energy value, and a minimum of the first minimum free energy value and the second minimum free energy value. In some embodiments, the method may further include comparing the determined difference to a target value.

In another aspect, the present disclosure is directed to a method in a computer system for determining the presence or absence of a target nucleic acid sequence in a sample. The method includes determining a first free energy contribution parameter for a comparison of a first nucleic acid probe base sequence to a first plurality of target bases of a target sequence.

The method may include comparing the first free energy contribution parameter to a target value. In some embodiments, the method may further include generating a response based on the comparison to the target value.

In another aspect, the present disclosure is directed to a computer-readable memory medium containing instructions for controlling a computer processor to store in a data repository a data structure representing a comparison of a first plurality of nucleic acids with at least a second plurality of nucleic acids, by: determining one or more duplex interactions formed between the first plurality of nucleic acids and the at least second plurality of nucleic acids; and storing sets of thermodynamic values indicative of each of the one or more duplex interactions formed between the first plurality of nucleic acids and the at least second plurality of nucleic acids. In some embodiments, the duplex interactions are selected from dangling ends of two or more bases, unpaired single strands of two or more bases adjacent to a Watson-Crick base pairing, unpaired single strands of one or more bases adjacent to a non-Watson-Crick base pairing, tandem base pair mismatches of two or more bases, length-dependent terminal mismatches of nucleic acid base, terminal base pair mismatches, Watson-Crick base pairings, single base pairings of mismatched doublets, initial binding processes, or combinations thereof.

In another aspect, the present disclosure is directed to a computer readable storage medium storing instructions that, when executed on a computer, execute a method for determining thermodynamic characteristics of nucleic acid sequences. The method includes retrieving from storage one or more thermodynamic parameters associated with a binding comparison of a first nucleic acid base sequence to a first region of at least a second nucleic acid base sequence. The method may further include retrieving from storage one or more thermodynamic parameters associated with a binding comparison of the first nucleic acid base sequence to a second region of the at least second nucleic acid base sequence, the second region different from the first region by at least one nucleic acid base position along a nucleic acid sequence of the second nucleic acid base sequence.

In some embodiments, the one or more thermodynamic parameters comprise at least one of a dangling end of two or more bases thermodynamic parameter, an unpaired single strand of two or more bases adjacent to a Watson-Crick base pairing thermodynamic parameter, an unpaired single strand of one or more bases adjacent to a non-Watson-Crick base pairing thermodynamic parameter, a tandem base pair mismatch of two or more bases thermodynamic parameter, a length-dependent terminal mismatch of nucleic acid base thermodynamic parameter, and a terminal base pair mismatch thermodynamic parameter.

In another aspect, the present disclosure is directed to a computing device for evaluating thermodynamic properties of a nucleic acid probe and a target nucleic acid sequence. The device includes an integrated circuit, an input device, and a processor. In some embodiments, the integrated circuit includes a plurality of logic components. In some embodiments, the input device is coupled to the integrated circuit and is operable to provide data indicative of one or more thermodynamic characteristics of a comparison of individual base pair binding events associated with a nucleic acid probe and at least a first region of a nucleic acid sequence.

In some embodiments, the processor is coupled to the integrated circuit and is operable to analyze an output of one or more of the plurality of logic components and to determine a thermodynamic free energy of the comparison of the individual base pair binding events associated with the nucleic acid probe and the at least first region of the nucleic acid sequence.

In yet another aspect, the present disclosure is directed to a method for analyzing a genomic sequence. The method includes identifying a genetic region in the genomic sequence characterized by at least one nucleic acid sequence. The method may include providing a first probe and at least a second probe, the first and the at least second probes provided based on a free energy gap characteristic indicative of a binding affinity for the at least one nucleic acid sequence. The method may further include detecting whether a binding event between the first and the at least second probes and the at least one nucleic acid sequence has occurred.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements, as drawn, are not intended to convey any information regarding the actual shape of the particular elements, and have been solely selected for ease of recognition in the drawings.

FIG. 1 is a schematic diagram of a data processing system for analyzing a biological sample according to one illustrative embodiment.

FIG. 2A is an illustration of one possible duplex formed by two nucleic acid sequences each comprising nine bases according to one illustrative embodiment.

FIGS. 2B and 2C are thermodynamic equation parameters associated with various duplex interactions formed by the two nucleic acid sequences of FIG. 2A according to multiple illustrative embodiments.

FIG. 3A is an illustration of a relative alignment of a long sequence (e.g., a DNA sequence) and a short sequence (e.g., a 16-base DNA sequence) according to one illustrative embodiment.

FIG. 3B is an illustration of a sliding window frame for a relative alignment of the long and short sequences of FIG. 3A according to one illustrative embodiment.

FIG. 4 is a schematic diagram of a portion of a circuitry including three nearest neighbor (n-n) doublets in a logic device according to one illustrative embodiment.

FIG. 5 is an illustration of an in-series calculation scheme for a relative alignment of a long sequence (e.g., a DNA sequence), and a short sequence (e.g., a 14-base DNA sequence) according to one illustrative embodiment.

FIG. 6 is an illustration of an in-parallel calculation scheme for a relative alignment of a long sequence (e.g., a DNA sequence), and a short sequence (e.g., a 14-base DNA sequence) according to one illustrative embodiment.

FIG. 7 is a schematic diagram of a pipelining implementation technique for enabling multiple alignment calculations to be performed on, for example, a circuit for thermodynamic comparisons of sequences according to one illustrative embodiment.

FIG. 8 is an exemplary screen display for a data processing system for analyzing a biological sample according to one illustrative embodiment.

FIG. 9 is Hybridization Intensity versus Time plot for perfect match and single base pair mismatch duplexes according to one illustrative embodiment. Probe and target sequences are shown in the inset.

FIG. 10 is a flow diagram of a method in a computer system for analyzing nucleic acid probes according to one illustrative embodiment.

FIG. 11 is a flow diagram of a method in a computer system for determining the presence or absence of a target nucleic acid sequence in a sample according to one illustrative embodiment.

FIG. 12 a flow diagram of a method for analyzing a genomic sequence according to one illustrative embodiment.

FIG. 13 is a flow diagram of a method for determining the thermodynamic characteristics of nucleic acid sequences according to one illustrative embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with computing systems including, processors, memories, and/or buses have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “in another embodiment,” or “in some embodiments” means that a particular referent feature, structure, or characteristic described in connection with the embodiment is included in at least one embodiment. Thus, the appearance of the phrases “in one embodiment,” or “in an embodiment,” or “in another embodiment,” or “in some embodiments” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to computing device including a “controller” includes a single controller, or two or more controllers. It should also be noted that the term “or” is generally employed in its sense including “and/or” unless the content clearly dictates otherwise.

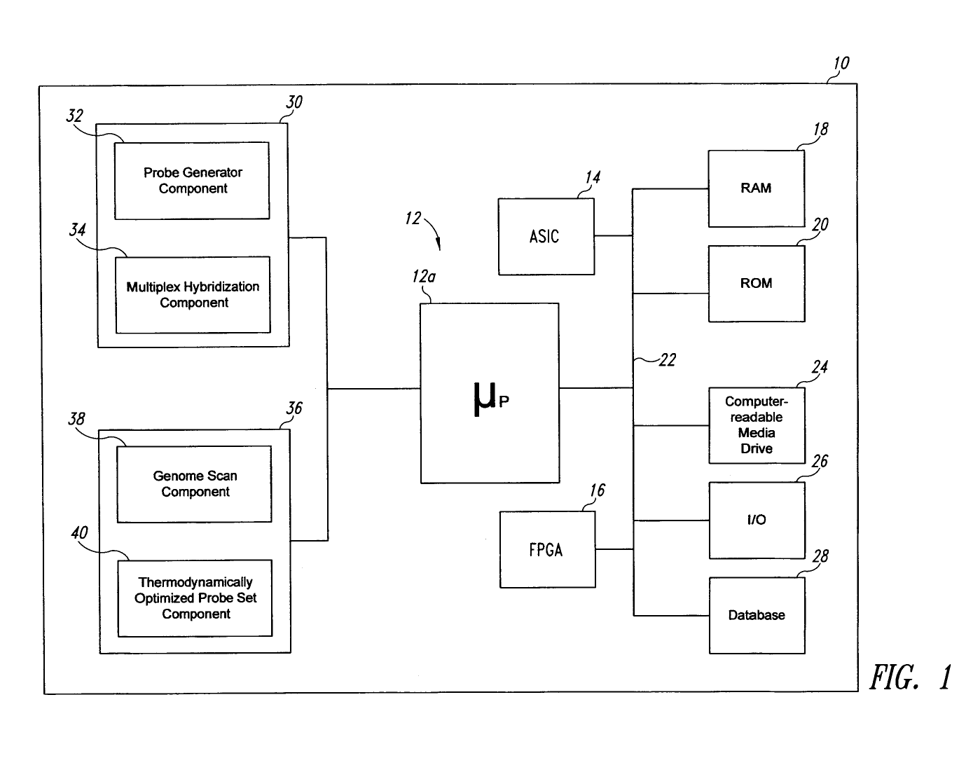

FIG. 1 shows a block diagram of a computing system 10 suitable for analyzing biological samples, analyzing nucleic acid probes, evaluating thermodynamic properties of nucleic acid sequences, or the like. The computing system 10 may include one or more controllers 12 such as a microprocessor 12a, a central processing unit (CPU) (not shown), a digital signal processor (DSP) (not shown), an application-specific integrated circuit (ASIC) 14, a field programmable gate array 16, or the like, or combinations thereof, and may include discrete digital and/or analog circuit elements or electronics.

The computing system 10 may further include one or more memories that store instructions and/or data, for example, random access memory (RAM) 18, read-only memory (ROM) 20, or the like, coupled to the controller 12 by one or more instruction, data, and/or power buses 22. The computing system 10 may further include a computer-readable media drive or memory slot 24, and one or more input/output components 26 such as, for example, a graphical user interface, a display, a keyboard, a keypad, a trackball, a joystick, a touch-screen, a mouse, a switch, a dial, or the like, or any other peripheral device. The computing system 10 may further include one or more databases 28.

The computer-readable media drive or memory slot 24 may be configured to accept computer-readable memory media. In some embodiments, a program for causing the computer system 10 to execute any of the disclosed methods can be stored on a computer-readable recording medium. Examples of computer-readable memory media include CD-R, CD-ROM, DVD, data signal embodied in a carrier wave, flash memory, floppy disk, hard drive, magnetic tape, magnetooptic disk, MINIDISC, non-volatile memory card, EEPROM, optical disk, optical storage, RAM, ROM, system memory, web server, or the like.

In some embodiments, the computing system 10 is configured to compare an input associated with the biological sample to a database 28 of stored reference values, and to generate a response based in part on the comparison. In some embodiments, the computing system 10 is provided for analyzing hybridization of target molecules to probes on substrate-bound nucleic acid, peptide, or protein arrays. In some embodiments, the computing system 10 comprises a data processing system for analyzing a biological sample.

In some embodiments, the computing system 10 may include computer-readable memory media in the form of one or more logic devices (e.g., programmable logic devices, complex programmable logic device, field-programmable gate arrays, application specific integrated circuits, and the like) comprising one or more look-up tables.

In some embodiments, one or more of the disclosed methods can be implemented using a memory medium in which executable instructions or software for realizing the functions, or implementing one or more of the instructions of the various disclosed embodiments, have been stored and are supplied to the computer system 10 or a component of the computer system 10 such as, for example, a micro processor unit, or central processing unit, or the like of the computer system 10. For example, in some embodiments, the computer system 10, or a component thereof, reads and executes executable instructions stored in a memory medium. In some embodiments, the executable instructions themselves read from the memory medium and realize the various functions of one or more of the disclosed embodiments. The computing system 10 is also suitable for implementing one or more of the disclosed methods and/or instructions associated with one or more of the embodiments comprising computer-readable media.

In some embodiments, a computer-readable memory medium includes instructions for controlling a computer processor to store in a data repository a data structure with data representing a comparison of a first plurality of nucleic acids with at least a second plurality of nucleic acids. In some embodiments, the instructions include determining one or more duplex interactions formed between the first plurality of nucleic acids and the at least second plurality of nucleic acids. In some embodiments, the instructions include instructions associated with storing sets of thermodynamic values indicative of each of the one or more duplex interactions formed between the first plurality of nucleic acids and the at least second plurality of nucleic acids.

In some embodiments, the duplex interactions are selected from dangling ends of two or more bases, unpaired single strands of two or more bases adjacent to a Watson-Crick base pairing, unpaired single strands of one or more bases adjacent to a non Watson-Crick base pairing, tandem base pair mismatches of two or more bases, length-dependent terminal mismatches of nucleic acid base, terminal base pair mismatches, Watson-Crick base pairings, single base pairings of mismatched doublets, initial binding processes, or combinations thereof.

The computing system 10 may further include a probe-target analysis component 30 including a probe generator component 32 and a multiplex hybridization component 34.

The probe-target analysis component 30 is operable to, for example, thermodynamically compare sequences of pairs of DNA strands, determine the sequence dependent thermodynamic stability for each alignment of the strands, compare stabilities of different duplexes at each alignment with those of the desired perfect match duplexes, and find those pairs of strands likely to crosshybridize. The probe-target analysis component 30 uses thermodynamic-based screening of probes and targets, rather than text-based screening for determining cross-hybridization propensity.

As previously noted, most commercially available probe design strategies rely on text-based similarity alignment routines to identify and filter candidate probe sequences. In some embodiments, the probe-target analysis component 30 is operable to search, compare, and select sets of probe sequences based on thermodynamic parameters representative of the various duplex interactions. For example, the probe-target analysis component 30 is operable to search and/or compare probes based on, for example, thermodynamic characteristics associated with the probes, and to select sets of probes whose individual members differ in one or more thermodynamic characteristics from one another. Simplicity of the probe-target analysis component 30 defines its elegance and thereby enables machine programmability.

In some embodiments, the probe-target analysis component 30 is configured to provide optimal sets of probe sequences designed to bind to specific target sequences according to one or more of the following desired characteristics: (1) probes bind specifically to defined target sequences; (2) probes do not bind targets other than the desired ones; and (3) probes do not bind any other probes. Accordingly, optimal sets of DNA probe sequences for specific targets may be generated using any of the aforementioned desired characteristics.

For example, given a first nucleic acid probe (α) and a first target nucleic acid sequence (α′); and a second nucleic acid probe (β) and a second target nucleic acid sequence (β′) characteristics of the set {α, β} can be determined by comparing the thermodynamics of every pair of sequences, α and β, in the set as follows. (1) free-energy (ΔG) of the perfect match duplex formed from a with its target (α′). (2) minimum ΔG over all duplexes (at every possible alignment) formed between α and β's target (β′). (3) minimum ΔG over all duplexes formed from α and β. Generally, (1) will have a value much less (i.e., be more stable) than either (2) or (3).

A basic measure of the fitness of the set can be obtained by taking the difference between the maximum of all calculated values of (1) and the minimum value of all the (2) and (3) values. This difference is generally referred to as the energy “gap” between desired duplexes (each probe in a perfect match with its target) and undesired cross-hybrids. In some embodiments, the goal is to make this gap as large as possible. By searching sequences based on thermodynamics differences, rather that their text identity or mere sequence homology, the probe-target analysis component 30 is operable to find probe sequences that are highly specific for their desired targets and have the lowest probability of cross-hybridization.

In some embodiments, the probe-target analysis component 30 is operable to identify sequences that fall below a target binding threshold value. These sequences are deemed unacceptable, eliminated and replaced. Generated sets are then compared to the “best set so far”. If the most recent set is better, sequences within it replace the current set and become the “best set so far” to be compared against other sets. In some embodiments, this iterative procedure continues until a set that satisfies a target energy gap (e.g., that maximizes the energy gap) is obtained. The method also allows consideration of additional constraints on the generated sequences. For example, a target G-C percentage and thereby range of thermodynamic stability of the sequence sets can be specified. Lexical rules can also be imposed (e.g., not allowing certain sequence patterns, (CCC or GGG)). Thermodynamic constraints can also be imposed (e.g., probe:target complexes should have a melting temperature (tm) over 20° C.). Also, probes can be designed while considering the potential interactions with other sequences in the set. Generated sequences should not form a lower ΔG (i.e., more stable) duplex complex, with any of these other sequences (e.g., from the Human Genome). Constraints can be applied at, for example, the time initial or as replacement sequences are generated.

Duplex interactions between nucleic acid probes and targets are generally sequence dependent. Every nucleic acid probe strand present in a multiplex reaction binds, with finite propensity, to nucleic acid targets other than the perfect match complementary sequence target. The extent of binding between two single strands depends on the sequence dependent free-energy of the duplex that they form. The thermodynamics of, for example, short duplex DNAs can be determined (e.g., calculated) using, for example, the nearest neighbor (n-n) model.

Simulations have shown that cross-hybridization (targets binding to probes non-specifically) can have significant effects on hybridization reactions and their interpretation. Accordingly, probes designed with forethought to minimize cross-hybridization may produce more accurate hybridization tests. Minimizing cross-hybridization may involve, in some cases, searching sequences based on thermodynamics differences, rather that their text identity or mere sequence homology. Accordingly, a need exists for the ability to quickly and thermodynamically scan probes against the genome so assays can be designed to minimize cross-hybridization based on thermodynamic rules instead of text homology. Platforms needing high throughput and reliable probes such as, for example, DNA microarrays, real time PCR, and flow cytometry may benefit from a thermodynamic scanning tool capable of setting the scale for minimizing cross-hybridization with undesired regions.

In some embodiments, the computer system 10 takes the form of a computing device for evaluating thermodynamic properties of a nucleic acid probe and a target nucleic acid sequence. The computing device may include an integrated circuit an input device 26, and a controller 12 (e.g., a processor, and the like).

The integrated circuit may include a plurality of logic components. The input device 26 may be coupled to integrated circuit and may be operable to provide data indicative of one or more thermodynamic characteristics of a comparison of individual base pair binding events associated with a nucleic acid probe and at least a first region of a nucleic acid sequence.

In some embodiments, the processor is coupled to the integrated circuit, and is operable to analyze an output of one or more of the plurality of logic components and to determine a thermodynamic free energy of the comparison of the individual base pair binding events associated with the nucleic acid probe and the at least first region of the nucleic acid sequence.

In some embodiments, the integrated circuit comprises an application specific integrated circuit 14 having a plurality of predefined logic components. In some embodiments, the integrated circuit comprises a field programmable gate array 16 having a plurality of programmable logic components.

In some embodiments, the computing system 10 takes the form of a data processing system for analyzing a biological sample. For example, in some embodiments, the computing system 10 comprises a computer-readable memory medium comprising thermodynamic data configured as a data structure for use in analyzing biological samples.

The data structure may comprise a thermodynamic data section including thermodynamic data representative of dangling ends of two or more bases. In some embodiments, the thermodynamic data section may further include thermodynamic data representative of unpaired single strands of two or more bases adjacent to a Watson-Crick base pairing. In some embodiments, the thermodynamic data section may further include thermodynamic data representative of unpaired single strands of one or more bases adjacent to a non-Watson-Crick base pairing. In some embodiments, the thermodynamic data section may further include thermodynamic data representative of tandem base pair mismatches of two or more bases. In some embodiments, the thermodynamic data section may further include thermodynamic data representative of length-dependent terminal mismatches of nucleic acid bases. In some embodiments, the thermodynamic data section may further include thermodynamic data representative of terminal base pair mismatches.

In some embodiments, the thermodynamic data section may further comprise thermodynamic data representative of dangling ends of a single nucleic acid base, thermodynamic data representative of Watson-Crick base pairings, thermodynamic data representative of single base pairings of mismatched doublets, thermodynamic data representative of initial binding processes, or combinations thereof.

In some embodiments, the thermodynamic data comprises nearest-neighbor free energy values, nearest-neighbor enthalpy values, or nearest-neighbor entropy values, or combinations thereof. In some embodiments, the thermodynamic data comprises binding affinity data indicative of a nucleic acid base sequence binding affinity to a target, and stability data indicative of a thermodynamic stability of a nucleic acid base sequence bound to the target, or combinations thereof. In some embodiments, the thermodynamic data comprises salt concentration-dependent thermodynamic data, buffer concentration-dependent thermodynamic data, sample concentration-dependent thermodynamic data, temperature-dependent thermodynamic data, or combinations thereof.

In some other embodiments, the thermodynamic data section may include any combinations of the disclosed thermodynamic data.

In some embodiments, the computing system 10 includes a controller 12 configured to compare an input associated with the biological sample to the thermodynamic data, and to generate a response based on the comparison.

In some embodiments the controller 12 is configured to compare the input associated with the biological sample to the thermodynamic data, and to generate at least one of a comparison plot, comparison data, an indication of a level of gene expression, an indication of a presence or absence of one or more nucleic acid sequences, or an indication of an L-length-mer composition of a target DNA fragment based on the comparison.

Among inputs associated with the biological samples examples include at least of one of an output generated from a detected image of the biological sampled applied to an array, gene expression data, nucleic acid sequence data, an n-dimensional expression profile vector of the biological sample, a genome of an organism, or combinations thereof.

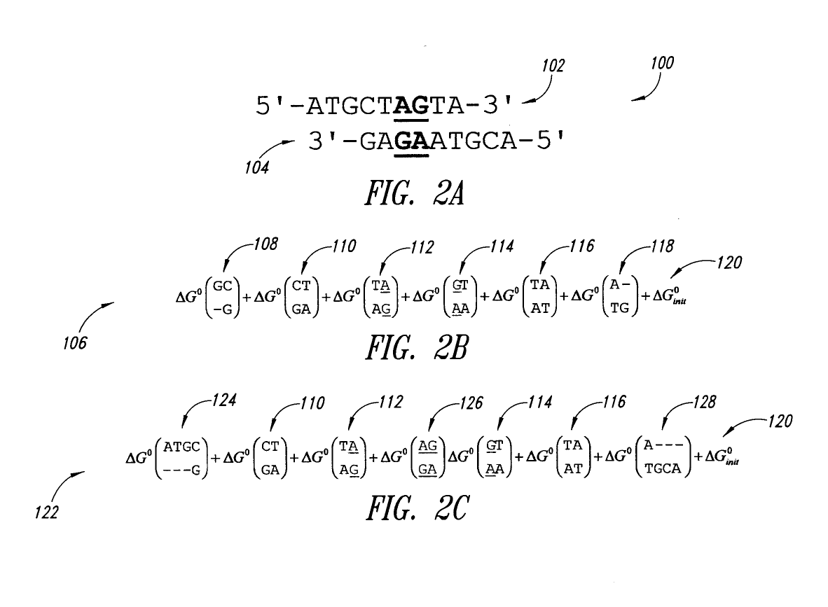

FIG. 2A shows one of the many possible duplexes 100 formed by a first and a second nucleic acid sequence 102, 104 each comprising nine bases. As previously noted, much of computational genetics treats duplex formation as a binary decision, either the bases are complementary (A-T or C-G), or they are not. But in reality nucleic acid sequences often bind to other nucleic acid sequences that are similar to their corresponding complementary target sequence. For example, in some instances a nucleic acid may form a duplex with a sequence that is very different than that of its corresponding complementary target sequence, but that might have a thermodynamic stability that is “similar” in magnitude. Accordingly, the extents of binding of each duplex will be “similar”.

Two sequences may have multiple different sequence alignments in which a duplex of the two can form.

The term “sequence alignment” generally refers to a way of arranging or comparing the primary sequences of DNAs, RNAs, or proteins to identify regions of similarity that may be a consequence of functional, structural, or evolutionary relationships between the sequences. Aligned sequences of nucleotide or amino acid residues are typically represented as rows within a matrix. Gaps are inserted between the residues so that residues with identical or similar characters are aligned in successive columns.

In protein sequence alignment or comparison, the degree of similarity between amino acids occupying a particular position in the sequence can be interpreted as a rough measure of how conserved a particular region or sequence motif is among lineages. The absence of substitutions, or the presence of only very conservative substitutions (that is, the substitution of amino acids whose side chains have similar biochemical properties) in a particular region of the sequence, may suggest that this region has structural or functional importance. Although DNA and RNA nucleotide bases are more similar to each other than to amino acids, the conservation of base pairing can indicate a similar functional or structural role.

FIGS. 2B and 2C provide examples of how nearest-neighbor thermodynamic parameters are used to calculate the stability of hybrid duplexes.

For example, two 24-base oligomers may have as many as 47 different sequence alignments in which a duplex of the two can form. Each of these duplexes will have an associated energy of formation. One approach for assessing the thermodynamic parameters associated with duplex interactions formed between, for example, a first plurality of nucleic acids and at least a second plurality of nucleic acids employs the nearest-neighbor thermodynamic model.

Based on the nearest-neighbor thermodynamic model, the energy of duplex formation is determined by the bases of one sequence, taken in paired bases, along with the paired bases of the mating sequence. Accordingly, the thermodynamic stability of two stranded complexes 100 is determined from the sum 106, 122 of n-n interactions over all n-n doublets in the duplex. An n-n doublet is comprised of two “base pair” units. A doublet can be, for example, a Watson-Crick hydrogen bonded base pair 110, a single 112 or double mismatch base pair 126, or the like. Thermodynamic stability of both is sequence dependent. Thus, each n-n doublet can be comprised of two Watson-Crick base pairs 110. An n-n doublet can contain one Watson-Crick base pair and one mismatch base pair (a single base pair mismatch) 116, 112. An n-n doublet can also be comprised of two mismatch base pairs, in a so-called tandem mismatch 126.

The nearest-neighbor thermodynamic model approach may include, for example, determining thermodynamic data representative of: dangling ends of a single nucleic acid base 108, 118; Watson-Crick base pairings 110;116; single base pairings of mismatched doublets 112, 114; initial binding processes 120; unpaired single strands of two or more bases adjacent to a Watson-Crick base pairing 124, 128; tandem base pair mismatches of two or more bases 126; dangling ends of two or more bases; single strands of one or more bases adjacent to a non Watson-Crick base pairing; terminal base pair mismatches; length-dependent terminal mismatches of nucleic acid base; or combinations thereof.

FIG. 2B illustrates an example of how thermodynamic parameters are used to predict duplex DNA stability. Parameter values for single base pair dangling ends 108, 118, perfect match Watson-Crick base pair doublets 110, 116, and single base pair mismatches are employed 112, 114. In this approach, the ΔG of tandem mismatches is approximated by only considering the single mismatch ΔG values for the particular mismatches adjoining a Watson-Crick base pair. Often contributions of tandem mismatches containing more than two mismatch base pairs are completely ignored or approximated by generic loop thermodynamic parameters.

FIG. 2C illustrates an example of an approach that accounts for, among other things, n-n sequence dependent interactions for Watson-Crick base pair doublets and doublets containing single base pair mismatches. A more detailed approach also explicitly includes considerations of tandem mismatches 126 and sequence dependent single strand dangling ends longer than a single base 124, 128. In some embodiments, a length dependent term for duplex initiation is also included. In some embodiments, the n-n model representation and corresponding sequence dependent parameters of thermodynamic DNA stability can be stored as, for example, data tables.

Traditionally, the nearest-neighbor (n-n) model generally assumes that the stability of a duplex DNA depends on the identity and orientation of neighboring base pairs. Any Watson-Crick DNA duplex structure will have ten possible n-n interactions. These interactions are:

$$( \begin{matrix}AA \\ TT\end{matrix} ) , ( \begin{matrix}AT \\ TA\end{matrix} ) , ( \begin{matrix}TA \\ AT\end{matrix} ) , ( \begin{matrix}CA \\ GT\end{matrix} ) , ( \begin{matrix}CG \\ GC\end{matrix} ) , ( \begin{matrix}GC \\ CG\end{matrix} ) , ( \begin{matrix}CT \\ GA\end{matrix} ) , ( \begin{matrix}GT \\ CA\end{matrix} ) , ( \begin{matrix}GA \\ CT\end{matrix} ) , and ( \begin{matrix}GG \\ CC\end{matrix} ) .$$

The stability of a DNA duplex may be predicted from its primary sequence if the relative stability (ΔG^{o}) of each DNA n-n interaction is known. It is these n-n parameters, when cast in the same format, that are in general agreement amongst the various laboratories. In practice, however, there are many other duplex interactions not accounted for by the n-n model such as those disclosed herein that should also be considered in the thermodynamic description of duplex DNA.

The total free energy change of the DNA helix from its individual strands is given by:

ΔG^{o}(total)=ΣᵢnᵢΔGo(i)+ΔG^{o}(init w/term GC)+ΔG^{o}(init w/term AT)+ΔG^{o}(sym)

where ΔG^{o}(i) are the strand free energy changes for the ten possible Watson-Crick n-n's, nᵢ is the number of occurrences of each nearest neighbor, i, and ΔG^{o}(sym) equals +0.43 kcal/mol if the duplex is self-complementary and zero if it is not self-complementary. To account for differences between duplexes with terminal AT versus terminal GC pairs, two initiation parameters are introduced.

Some probe design strategies may also apply several empirical factors that make certain “corrections” to the calculated thermodynamics. For example, a parabolic n-n model, in which n-n ΔG values are weighted by an upward parabolic function centered at the middle and increasing at the ends, where as the n-n doublets approach the ends they become less stable (have higher ΔG values).

Although some nearest-neighbor parameters for single base pair mismatches for various possible nearest-neighbor combinations are known, there are no known parameter sets for tandem mismatches.

In some embodiments, the thermodynamic transition parameters, ΔH, ΔS, and ΔG, used in kinetic and equilibrium model calculations, may be determined from sequence-dependent thermodynamic parameters. See e.g., Benight et al., “Statistical Thermodynamics and Kinetics of DNA Multiplex Hybridization Reactions” Biophys J., 91(11), pp. 4133-4153 (2006).

Consider, for example, the hybrid duplex formed by sequences 5′-AGCGATGA-3′- and -3′-CAATAATT-5′ and its decomposition into nearest-neighbor components of the enthalpy, ΔH (mismatches are underlined):

$$Δ H ( \begin{matrix}AGCGATGA - \\ - CAATAATT\end{matrix} ) = Δ H ( \begin{matrix}AG \\ - C\end{matrix} ) + Δ H ( \begin{matrix}G \underline{C} \\ C \underline{A}\end{matrix} ) + Δ H ( \begin{matrix}\underline{CG} \\ \underline{AA}\end{matrix} ) + Δ H ( \begin{matrix}\underline{G} A \\ \underline{A} T\end{matrix} ) + Δ H ( \begin{matrix}AT \\ TA\end{matrix} ) + Δ H ( \begin{matrix}T \underline{G} \\ A \underline{A}\end{matrix} ) + Δ H ( \begin{matrix}\underline{G} A \\ \underline{A} T\end{matrix} ) + Δ H ( \begin{matrix}A - \\ TT\end{matrix} ) + Δ {H}_{init} ( \begin{matrix}A \\ T\end{matrix} ) + Δ {H}_{init} ( \begin{matrix}G \\ C\end{matrix} ) \qquad ( eq . 1 )$$

This duplex contains eight nearest-neighbor interactions, including single-base 5′ dangling ends. The nearest-neighbor dependent parameters

$$Δ H ( \begin{matrix}AG \\ - C\end{matrix} ) , Δ H ( \begin{matrix}G \underline{C} \\ C \underline{A}\end{matrix} ) , Δ H ( \begin{matrix}\underline{G} A \\ \underline{A} T\end{matrix} ) , Δ H ( \begin{matrix}AT \\ TA\end{matrix} ) , and the like$$

for the appropriate sequences and interactions are summarized in the following Tables 2-4.

TABLE 2
Nearest-neighbor thermodynamic parameters for w/c doublets.

| W/C doublet | Enthalpy(cal/mol) | Entropy (cal/kmol) |
|---|---|---|
| AA | −7900 | −22.2 |
| AC | −8400 | −22.4 |
| AG | −7800 | −21.0 |
| AT | −7200 | −20.4 |
| CA | −8500 | −22.7 |
| CC | −8000 | −19.9 |
| CG | −10,600 | −27.2 |
| GA | −8200 | −22.2 |
| GC | −9800 | −24.4 |
| TA | −7200 | −21.3 |

TABLE 3
Sequence-dependent thermodynamic parameters for dangling ends

| Dangling end | Enthalpy (cal/mol) | Entropy (cal/Kmol) |
|---|---|---|
| TA/-T | −6900 | −20.0 |
| AC/-G | −6300 | −17.1 |
| CA/G- | −5900 | −16.5 |
| GT/-A | −4200 | −15.0 |
| CT/G- | −5200 | −15.0 |
| GC/-G | −5100 | −14.0 |
| TG/-C | −4900 | −13.8 |
| AG/T- | −4100 | −13.1 |
| -C/TG | −4400 | −13.1 |
| CT/-A | −4100 | −13.0 |
| CC/-G | −4400 | −12.6 |
| AT/T- | −3800 | −12.6 |
| CG/-C | −4000 | −11.9 |
| -C/GG | −3900 | −11.2 |
| GG/-C | −3900 | −10.92 |
| TC/-G | −4000 | −10.9 |
| CG/G- | −3200 | −10.4 |
| AG/-C | −3700 | −10 |
| AT/-A | −2900 | −7.6 |
| CC/G- | −2600 | −7.4 |
| -C/AG | −2100 | −3.9 |
| TG/A- | −1600 | −3.6 |
| GA/-T | −1100 | −1.6 |
| AA/T- | −500 | −1.1 |
| TA/A- | −700 | −0.8 |
| TT/-A | −200 | −0.5 |
| -C/CG | −200 | −0.1 |
| AA/-T | 200 | 2.3 |
| CA/-T | 600 | 3.3 |
| TT/A- | 2900 | 10.4 |
| AC/T- | 4700 | 14.2 |
| TC/A- | 4400 | 14.9 |

TABLE 4
Sequence-dependent thermodynamic parameters
for single base pair mismatches

| MM | Enthalpy (cal/mol) | Entropy (cal/Kmol) |
|---|---|---|
| GC/GG | −6000 | −15.8 |
| CT/GT | −5000 | −15.8 |
| CG/GG | −4900 | −15.3 |
| GC/TG | −4400 | −12.3 |
| CG/GT | −4100 | −11.7 |
| AG/GC | −4000 | −13.2 |
| AG/TG | −3100 | −9.5 |
| AC/AG | −2900 | −9.8 |
| CT/GG | −2800 | −8.0 |
| AT/TT | −2700 | −10.8 |
| AT/TG | −2500 | −8.3 |
| GT/CT | −2200 | −8.4 |
| CC/GC | −1500 | −7.2 |
| CG/TC | −1500 | −6.1 |
| TT/AG | −1300 | −5.3 |
| AT/TC | −1200 | −6.2 |
| AG/AC | −900 | −4.2 |
| CC/GT | −800 | −4.5 |
| AC/CG | −700 | −3.8 |
| AG/TA | −700 | −2.3 |
| CA/GG | −700 | −2.3 |
| AA/TG | −600 | −2.3 |
| GA/CG | −600 | −1.0 |
| TA/GT | −100 | −1.7 |
| AC/TC | 0 | −4.4 |
| TA/TT | 200 | −1.5 |
| AC/GG | 500 | 3.2 |
| AG/CC | 600 | −0.6 |
| AC/TT | 700 | 0.2 |
| GA/AT | 700 | 0.7 |
| AG/TT | 1000 | 0.9 |
| TT/AC | 1000 | 0.7 |
| AA/TA | 1200 | 1.7 |
| TA/CT | 1200 | 0.7 |
| GA/GT | 1600 | 3.6 |
| CA/GC | 1900 | 3.7 |
| AA/TC | 2300 | 4.6 |
| GC/CT | 2300 | 5.4 |
| AA/GT | 3000 | 7.4 |
| GG/CT | 3300 | 10.4 |
| CA/AT | 3400 | 8.0 |
| CC/CG | 3600 | 8.9 |
| GT/TG | 4100 | 9.5 |
| AA/AT | 4200 | 12.9 |
| CC/AG | 5200 | 14.2 |
| CC/TG | 5200 | 13.5 |
| GA/CC | 5200 | 14.2 |
| AC/TA | 5300 | 14.6 |
| GG/TT | 5800 | 16.3 |
| CA/CT | 6100 | 16.4 |
| AA/TC | 7600 | 20.2 |

In some embodiments, initiation factors such as, for example,

$$Δ {H}_{init} ( \begin{matrix}A \\ T\end{matrix} ) and Δ {H}_{init} ( \begin{matrix}G \\ C\end{matrix} )$$

may be assigned values depending on the particular identities of the end base pairs. Values for the initiation thermodynamic parameters associated with the duplex formed by the 5′-AGCGATGA-3′- and -3′-CAATAATT-5′ sequences are as follows:

$$\begin{matrix}\begin{matrix}Δ {H}_{init} ( \begin{matrix}G \\ C\end{matrix} ) = 0.1 kcal / mol , Δ {S}_{init} ( \begin{matrix}G \\ C\end{matrix} ) \\ = - 2.8 kcal {K}^{- 1} {mol}^{- 1} ;\end{matrix} & ( eq . 2 ) \\ and & \\ \begin{matrix}Δ {H}_{init} ( \begin{matrix}A \\ T\end{matrix} ) = 2.3 kcal / mol , Δ {S}_{init} ( \begin{matrix}A \\ T\end{matrix} ) \\ = 4.1 kcal {K}^{- 1} {mol}^{- 1} .\end{matrix} & ( eq . 3 )\end{matrix}$$

The formulas for total free energy include:

ΔG=ΔH−TΔS (eq. 4);

Tₘ=ΔH/ΔS (eq. 5); and

ΔG=ΔH(1−T/Tₘ) (eq. 6).

In some embodiments, tandem mismatches are evaluated in terms of n-n contributions. In this approach tandem mismatch (mm) base pairs are assigned a ΔG value relative to the corresponding Watson-Crick base pair doublet values. See e.g., Benight et al., “Statistical Thermodynamics and Kinetics of DNA Multiplex Hybridization Reactions” Biophys J., 91(11), pp. 4133-4153 (2006). For example, the free-energy of a mismatch base pair doublet in a tandem mismatch complex can be assigned according to

ΔGₘₘ=κΔG_{PM}=κ(ΔH_{PM}−TΔS_{PM}) (eq 7),

where ΔG_{PM}, ΔH_{PM}, ΔS_{PM}, are the free energy, enthalpy, and entropy, respectively, for melting a hydrogen-bonded Watson-Crick base pair doublet. The factor κ is introduced as a means of scaling values of thermodynamic parameters of mismatch base pairs in tandem mismatches as a relative fraction of the stability of Watson-Crick perfect matches. The factor κ may be a single factor or one or more matrices of factors. In some embodiments, tandem mismatches can either be assumed to be minimal, κ=0, or assigned a K value of greater than zero (0) or less than or equal to one (1) (e.g., κ=0.5). Although consideration of tandem mismatches in this manner is clearly an oversimplified generalization, it provides a convenient means of universally weighting non-Watson-Crick tandem mismatch pair interactions differently than Watson-Crick base pairs, and discerning potential effects of tandem mismatch stability on multiplex hybridization.

Examples of sequence dependent values of tandem mismatch thermodynamic parameters (κ) are summarized in Table 5.

TABLE 5
Tandem Mismatch Thermodynamic Parameters

| n-n Tandem Mismatch | Δ G̅° (kcal/mol) | κ |
|---|---|---|
| 100% R | −1.31 | 0.8 |
| 25-75% Y + R | −0.95 | 0.6 |
| 100% Y | −0.32 | 0.2 |

The tandem mismatches values in Table 5 are grouped according to their purine (R) and pyrimidine (Y) composition. As suggested by the values of κ, contributions of tandem mismatches to duplex stability are much larger than presently assumed.

In some embodiments, nearest-neighbor thermodynamic parameters, tandem mismatches contributions, as well as other thermodynamics parameter associated with duplex binding may be determine experimentally using, for example, differential scanning calorimetry (DSC) techniques, UV-Melting analysis, thermal denaturation techniques, optical absorbance versus temperature measurements, or the like.

For example, DNA duplex melting transitions may be evaluated by measurements of DSC melting curves using, for example, a Nano-II differential scanning calorimeter (Calorimetry Sciences Corp., Provo, Utah). In some embodiments, DSC data is collected as the change in excess heat capacity ΔCₚ versus temperature T. Heating rates may vary from about 15° C./hr to about 90° C./hr. The average buffer base line determined from multiple (usually more than three) scans of the buffer alone, is subtracted from these curves. The resulting base line corrected curve is then normalized to total DNA concentration and the calorimetric transition enthalpy ΔH_{cal} and entropy ΔS_{cal} are determined from the normalized, base line corrected ΔCₚ vs. T curve.

In some embodiments, at least three forward and reverse ΔCₚ versus T scans are made per experiment. For short DNA melting curves, it is generally assumed that ΔCₚ (Tᵢₙᵢₜᵢₐₗ)−ΔCₚ (T_{final})=0. This assumption has been generally validated by the few attempts to evaluate any excess ΔCₚ in melting reactions, and it has been found that the contribution and the associated temperature dependence of thermodynamic parameters is very small.

In some embodiments, thermodynamic parameters are evaluated by DSC. DSC offers some advantages over, for example, optical absorbance versus temperature measurements. These include: (1) model independent parameter evaluation; and (2) no need to measure concentration dependence of the melting transition temperature, tₘ. Because DSC melting experiments are collected at relatively higher strand concentrations than for absorbance melting experiments, higher strand concentrations lead to more duplex formation. As a result melting experiments can be conducted on shorter duplexes at lower salt concentration.

A factor of probe design strategies is the quantitative determination of the propensity for intramolecular hairpin formation in probe and target strands. Known routines primarily rely on version a RNA and DNA folding package known as M-FOLD (developed by Dr. Michael Zuker of the Institute for Biomedical Computing, Washington University School of Medicine).

Some embodiment of the disclosed approaches of comparing and selecting probes based on the largest differences in ΔG of desired versus undesired hybridizations, eliminate potential hairpin forming sequences, since two strands capable of forming hairpins are also self-complementary. Their sequence could also promote bi-molecular duplex formation instead of an internal single strand loop comprised of tandem mismatches. These are apparently effectively filtered by the probe-target analysis component 30 and in preliminary testing it has found that the probe-target analysis component 30 is also an effective “filter” of self-complementary sequences that might be expected to have the strongest probability of hairpin formation. Partitioning of DNA sequence dependent contributions to thermodynamic stability into n-n components is the only known higher order representation of DNA that is not text-based. The n-n model is also ideally suited for an electronic circuit designed to make calculations and comparisons between the thermodynamics of sequences in a repetitive manner, using a database of n-n parameters.

When determining whether or not a particular probe sequence will bind with a set of large target sequences (e.g., a genome), as well as where it will bind, the energy of the duplex at each alignment of the probe with each of the targets must be accounted for. For example, given a probe length of 24 bases, and a genome to be examined having on the order of 6 billion bases, over 600 billion arithmetic operations must be performed to determine all the low energy alignment points. Along with these arithmetic operations, a large number of control and data flow operations are also required.

The extent of computations means that it takes a relatively long time (on the order of an hour or more), for a general purpose computer to make this determination, and thus such computations may become a rate limiting step.

Integrated circuitry offers tremendous computation speed by allowing parallelization of repetitive calculations. Using the n-n model thermodynamic parameters for calculating duplex stability results in fast thermodynamic scans of long DNA sequences.

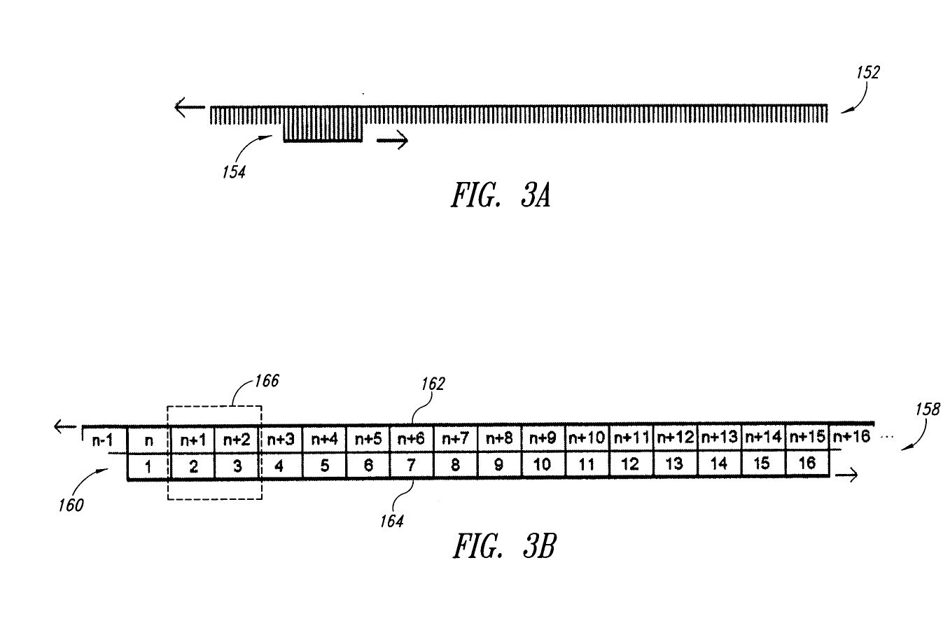

FIGS. 3A and 3B show the process of relative alignment for a long sequence 152, 158 (e.g., a DNA sequence, a genome) and a short sequences 154, 160 (e.g., a 16-base DNA sequence) as they are repetitively compared in a sliding window frame. Thermodynamic stabilities ΔG of the duplex in each alignment window are calculated in parallel as described below. In some embodiments, the ΔG values for the stable duplexes are saved in memory units for post-scan analysis. Duplex stabilities can be calculated at each configuration using, for example, the n-n model. For example, duplex stabilities can be calculated successive nearest neighbor (n-n) doublets 166. In some embodiments, aligning a first nucleic acid base 164 with a nucleic acid target base 162 includes shifting the first nucleic acid probe base sequence by at least one base in comparison to the plurality of target bases of the target sequence to define a second plurality of target bases, and determining the free energy contribution parameter for the comparison of the first nucleic acid probe base sequences with the second plurality of target bases. The “sliding window frame” concept ignores sequences that have significant thermodynamic stability, but are not fully in the same “register.” For example, in some embodiments, nucleic acid sequences comprising mismatches that are disordered (i.e., sequences that form one or more bulges or asymmetric loops) may be out-of-register regarding its relative alignment to a corresponding duplex partner. These mismatches that are disordered may be treated in some embodiments, however, as disordered loops.

In some embodiments, aligning a first nucleic acid probe base with a plurality of target bases includes shifting the first nucleic acid probe base sequence by at least one base in comparison to the plurality of target bases of the target sequence to define a second plurality of target bases, and determining the free energy contribution parameter for the comparison of the first nucleic acid probe base sequences with the second plurality of target bases.

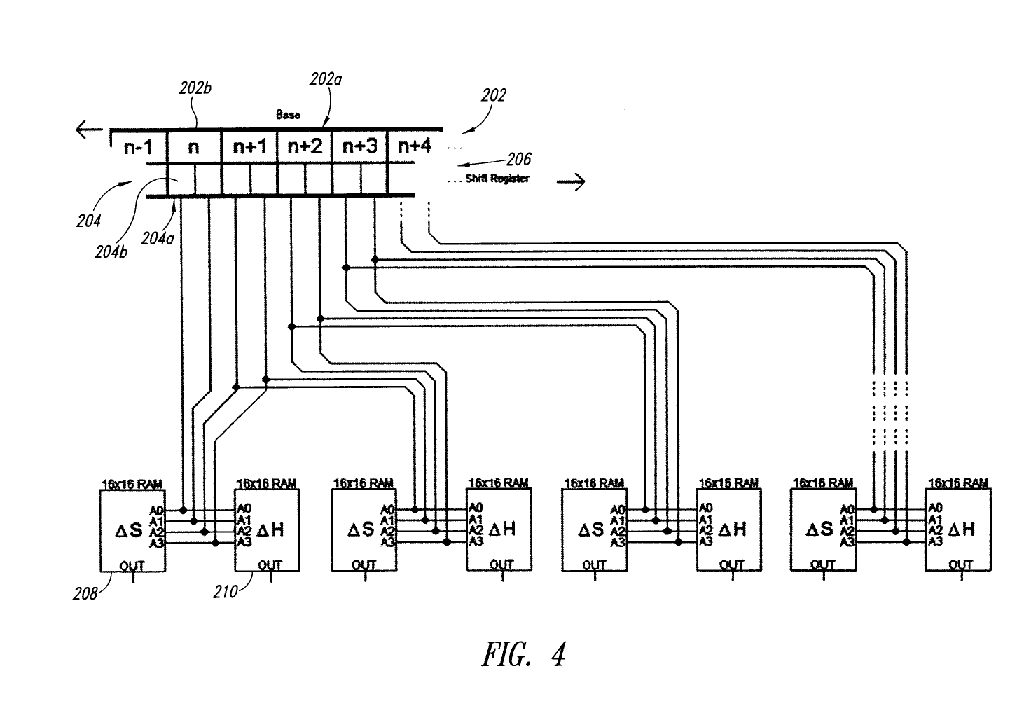

FIG. 4 show a schematic diagram representative of a portion of a circuitry including two successive nearest neighbor (n-n) doublets in a logic device. A short single strand query probe 202 is compared to a longer fragment 204 by repetitively sliding the shorter fragment 202 along the longer 204 and computing the thermodynamic stability (ΔG) of the duplex at each alignment position. In some embodiments, ΔG values for the stable duplexes are saved in memory units for post-scan analysis. In some embodiments, each pair of bases in a shift register 206 is addresses two RAM blocks (e.g., two 16×16 RAM Blocks 208, 210). Depending on the controller 12, common bus widths of 8, 16, 32, 64 bits, or the like may be used. In some embodiments, the bus width and the number of storage locations may vary.

The computing system 10 may include at least one memory interface component including one or more of sets of shift registers 206 interconnected in series or in parallel, or combinations thereof. In some embodiments, at least one shift register 202b, 204b of the one or more sets of shift registers 206 may be configured to receive a clock signal having a shift frequency. In some embodiments, the at least one shift register is capable of shifting data loaded into the shift register to a next one of the shift registers in the set 206 according to shift frequency. In some embodiments, thermodynamic data from a computer-readable memory medium is loaded into a corresponding shift register in the sets of shift registers 206 and the loaded thermodynamic data is shifted from the shift register to a next one of the shift registers in the set according to the clock signal, such that the shift register maintains its shift frequency during any loading of the thermodynamic data.

The values addressed correspond to n-n parameters for ΔH and ΔS. All values must be added to give a single ΔS and ΔH for a given alignment, used to calculate the ΔG for that alignment (ΔG=ΔH−TΔS). The 16×16 Ram Blocks 208, 210 shown in FIG. 4 store the n-n thermodynamic parameter values accessed by the circuitry to compute thermodynamic stabilities, ΔG. The circuit compares an n-n doublet and selects the appropriate parameter from the table based on the identity of the particular n-n doublet encountered. In practice, a Ram Block 208, 210 is present for each doublet so each computation can be done simultaneously and sent into a pipelining scheme as will be described below.

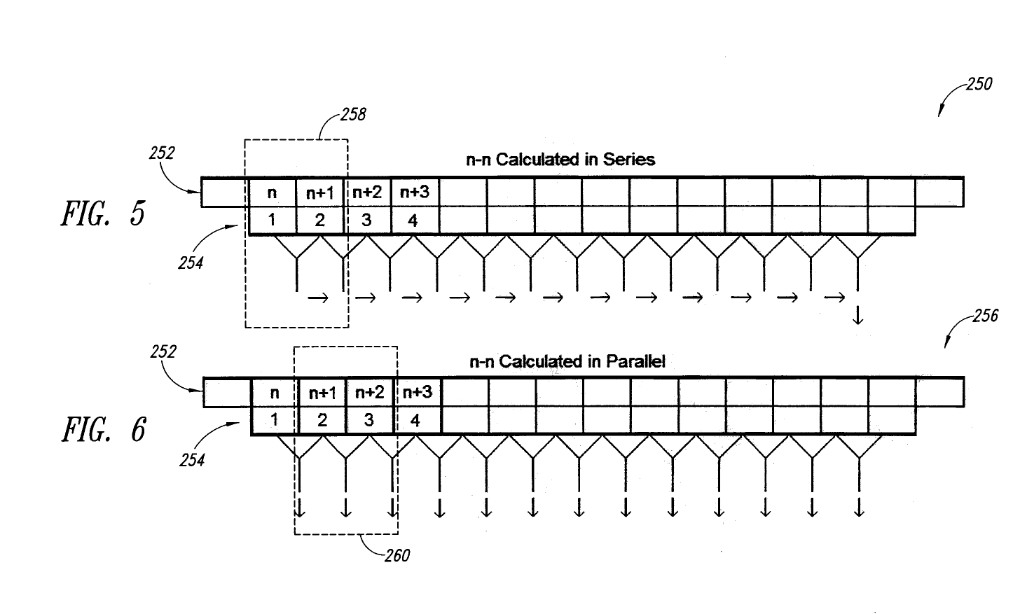

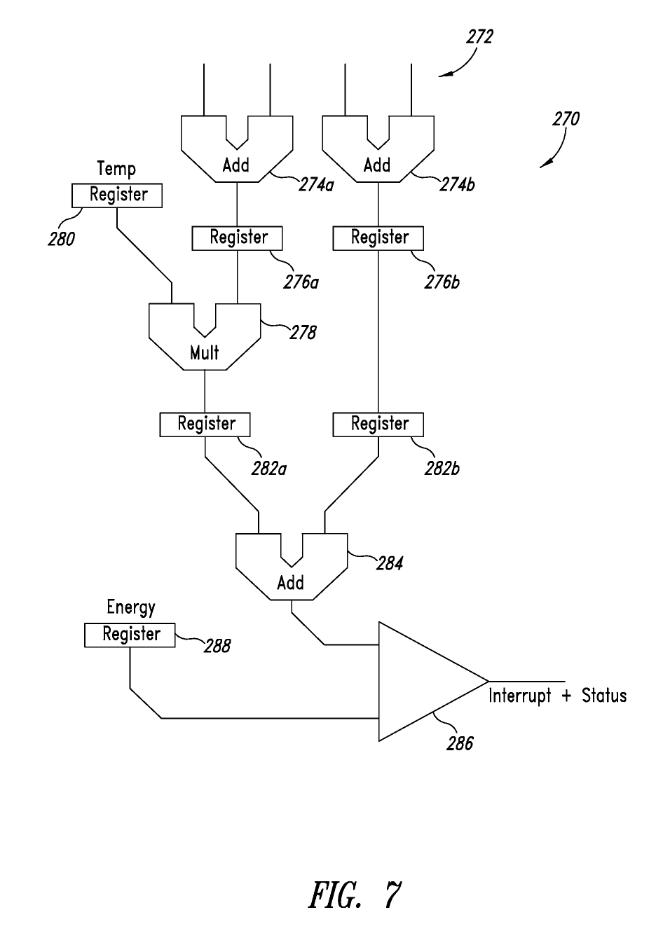

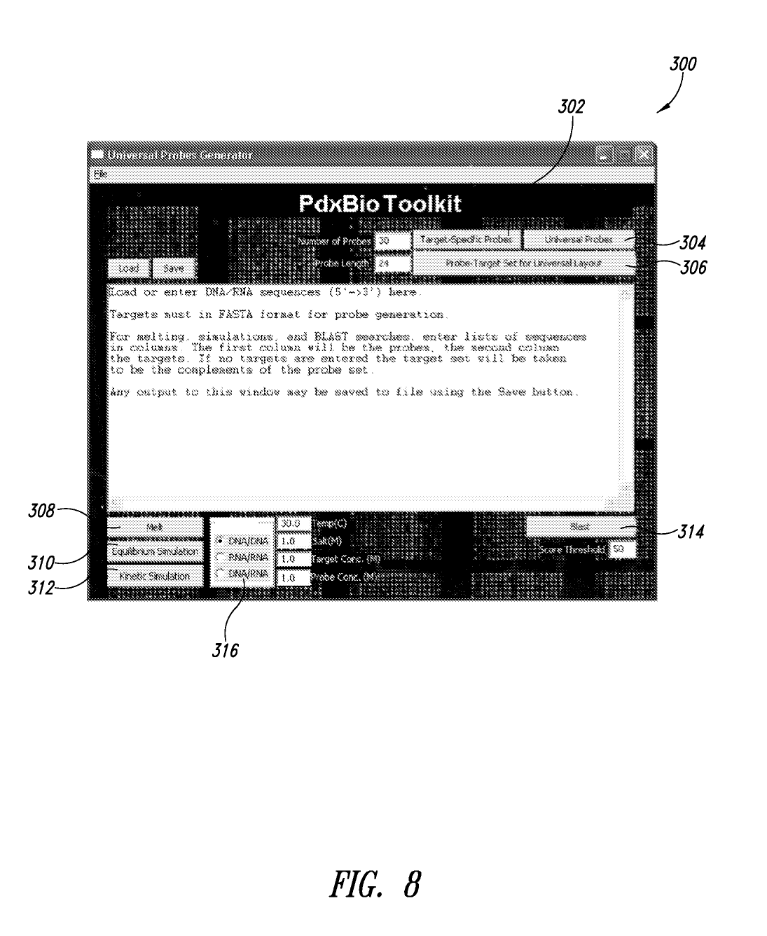

FIGS. 5 and 6 illustrate in-series 250 and in-parallel 256 calculation schemes, respectively, for a relative alignment of a long sequence 252 (e.g., a DNA sequence), and a short sequence 254 (e.g., a 14-base DNA sequence).

In some embodiments, the computing system 10 may simultaneously address all n-n elements that are stored in pairs of RAM Blocks 208, 210. As previously noted an n-n doublet 258, 260 is comprised of two “base pair” units. In some embodiments, there is one RAM Block 208, 210 per base pair. Accessed values may be sent into pipeline for calculation. This approach may significantly increase the computation speed of a comparison of a first plurality of nucleic acids with at least a second plurality of nucleic acids.

FIG. 7 shows a pipelining schema 270. The pipelining schema 270 is operable to, among other things, store and funnel data, as well as systematically add the elements with each clock cycle, resulting in a single ΔG value. Calculated ΔG values are compared to a reference free-energy, ΔG_{ref} that dictates whether the calculated ΔG of the probe/target complex is such that the complex poses a serious potential for cross-hybridization with other sequences. Pipelining enables multiple alignment calculations to be performed in the circuit at any instant thereby enabling increased throughput for thermodynamic comparisons of sequences.

At 272, the individual n-n elements are sent simultaneously to the pipeline 270. With each clock cycle, elements are added by adders 274a, 274b and may be buffered in registers 276a, 276b. A multiplier 278 may multiply a value representing the entropy (ΔS) by a value representing the temperature (T), which may be stored in a register 280. Resulting values may be buffered in registers 282a, 282b, before being added together by adder 284. The adder 284 adds the product (TΔS) to the enthalpy (ΔH) producing the free energy (ΔG=ΔH−TΔS). A comparator 286 compares the calculated ΔG value to a value that represents a reference free-energy ΔG_{ref} which may be stored in a register 288. The comparison dictates, for example, whether the probe of interest poses a threat for cross-hybridization at that alignment.

Referring to FIG. 4, in some embodiments, the computer system 10 may include a computer-readable memory medium and a shift register structure 206. The computer-readable memory medium may include thermodynamic data associated with at least one of a first nucleic acid sequence 202 and a second nucleic acid sequence 204. In some embodiments the thermodynamic data is configured as a data structure

In some embodiments, the shift register structure 206 may include a first set of shift registers 202a having a first plurality of shift registers 202b interconnected in series. In some embodiments, at least one of the first plurality of registers 202b is configured to receive a clock signal having a shift frequency. In some embodiments, the first set of shift registers 202a is configured to shift thermodynamic data associated with the first nucleic acid sequence 202 loaded into at least one shift register in the first set of shift registers 202a to a next one of a shift register in the first set of shift registers 202a according to, for example, the shift frequency.

The shift register structure 206 may further include a second set of shift register 204a having a second plurality of shift registers 204b interconnected in, for example, series. The second set of shift registers may include one or more shift register loaded with thermodynamic data associated with the second nucleic acid sequence 204.

In some embodiments, the shift register structure is configure to generate a comparison of thermodynamic data associated with the first nucleic acid sequence 202 loaded in one or more shift register in the first set of shift registers 202a and thermodynamic data associated with the second nucleic acid sequence 204 loaded in one or more shift register in the second set of shift registers 204a.

### EXAMPLE 1

### Estimates on Speed Advantages

An estimate on the enormous enhancements in speed that might be realized can be made with the following “back of the envelope” calculation. Bear in mind, however, that the following represents the optimum “theoretical” speed enhancement that can be obtained. What is actually obtained will, of course, depend on the functioning logic device circuitry. The algorithm makes thermodynamic comparisons serially and thus must compare all doublets in a probe-target duplex alignment before shifting the window by a base and making the same computation for the new probe-target duplex alignment. Thus, for a 17 base probe (n) scanned against a strand of the genome six billion base pairs in length (m), the algorithm must make (there are 16 n-n doublets formed in a 17 base pair duplex),

$$\begin{matrix}n \cdot m = 16 \frac{comparisons}{window} \cdot 6 \times {10}^{9} \frac{windows}{genome} \\ = 9.6 \times {10}^{10} \frac{comparisons}{genome} .\end{matrix} \qquad ( eq . 8 )$$

On a standard 3 GHz 1.6 Pentium the probe-target analysis component 30 can compare 600,000 bases per second (r). Thus a single 16 base probe can be scanned against the genome in, for example,

$$\begin{matrix}t = ( \frac{n \cdot m}{r} ) \\ = \frac{9.6 \times {10}^{10} \frac{comparisons}{genome}}{6 \times {10}^{5} \frac{comparisons}{sec}} \\ = 1.6 \times {10}^{5} \frac{sec}{genome} \\ = 44.4 \frac{hrs}{genome} .\end{matrix} \qquad ( eq . 9 )$$

Compare this to the disclosed systems and methods that makes calculations in parallel and therefore makes all comparisons for a single probe at once before shifting over by a base. The same number of comparisons has to be made; however, an FPGA, for example, uses its hardware logic gates and pipeline to effectively reduce the number of comparisons from 16 to 1 comparison per window cycle. Thus the same 17 base probe can be scanned against the same genome by making

$$\begin{matrix}n \cdot m = 1 \frac{comparisons}{window} \cdot 6 \times {10}^{9} \frac{windows}{genome} \\ = 6 \times {10}^{9} \frac{comparisons}{genome} .\end{matrix} \qquad ( eq . 10 )$$

Low end FPGAs process at 100 MHz, therefore the time for a scan of this 17 base probe against the genome is

$$\begin{matrix}t = ( \frac{n \cdot m}{r} ) \\ = \frac{6 \times {10}^{9} \frac{comparisons}{genome}}{100 \times {10}^{6} \frac{comparisons}{sec}} \\ = 60 \frac{sec}{genome} .\end{matrix} \qquad ( eq . 11 )$$

State of the art FPGAs process at 500 MHz which would allow scans five times faster. In this case the genomic scan would take 12 seconds to scan a 20-mer probe against a six billion base pair genome.

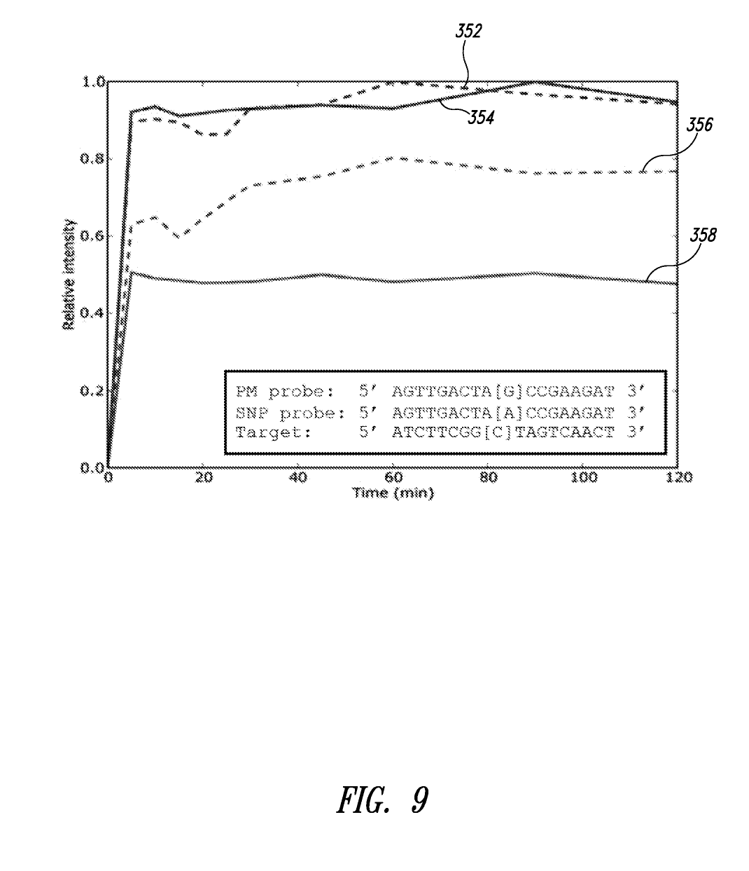

FIG. 8 shows exemplary screen display of graphical user interface 300 for a data processing system for analyzing a biological sample according to one illustrative embodiment. The graphical user interface 300 may include user selectable icons: designing target-specific probes from a list of target sequences 302; generating universal probes of a specified length from a long sequence entered 304; generating probe-target sets for universal probe layout 306; simulating melting data for a set of input sequences 308; simulating a full hybridization assay to equilibrium 310; simulating the kinetics of any reaction 312; performing BLAST searches 314; and supplying DNA/DNA, DNA/RNA, or RNA/RNA thermodynamic parameters 316. The probe-target analysis component 30 may also include BLAST capabilities as a means to perform homology searches for generated sets of sequences against a genome. Because BLAST searches are text-based and ineffective for the purpose of probe design, the probe-target analysis component 30 will, in some embodiments, employ one or more of the disclosed thermodynamically based approaches to selecting and/or generating probes.

### EXAMPLE 2

### Effectiveness of the Probe Generator Element

FIG. 9 shows a graph of Hybridization Intensities versus Time for perfect match 352, 354 and single base pair mismatch duplexes 356, 358. Probe and target sequences are shown in the inset. Results of hybridization experiments for two probes binding to a single target from two independent experiments 352, 356 and 354, 358, respectively, are displayed. The target sequence hybridizations to the PM probe form a perfect match duplex. Hybridizations of the target to the SNP probe results in a duplex containing a single base pair mismatch. Clear discrimination of a single base pair mismatch is obtained

The results illustrated in FIG. 9 provide an example that clearly demonstrates the efficacy of the probe-target analysis component 30 in designing optimum probes. In those studies, summarized in FIG. 9, probes were designed to simultaneously detect six different SNPs all in a single multiplex reaction. The target, T can form a duplex with each probe, P1 and P2. A T:P1 duplex is a perfect match duplex with all Watson-Crick base pairs. Duplex T:P2 however, is a duplex containing a single base pair mismatch (SNP). Eight different target strands were hybridized to microarrays containing 14 different probes (six probe pairs and two controls) located at different places on the microarray. At incubation times of 5, 10, 15, 20, 25, 30, 45, 60, 90 and 120 minutes a respective microarray was removed, washed, fixed, and read. Scanning and reading produced raw data in the form of signal intensity and background intensity values for each probe spot. Plots of the background corrected hybridization intensity versus time are shown in FIG. 9 for results from two independent experiments. Clear discrimination between the SNP and PM probes is obtained. Such discrimination in a multiplex environment attests to the utility and power of the probe-target analysis component 30 in the effective design of DNA probes for multiplex hybridization based assays.

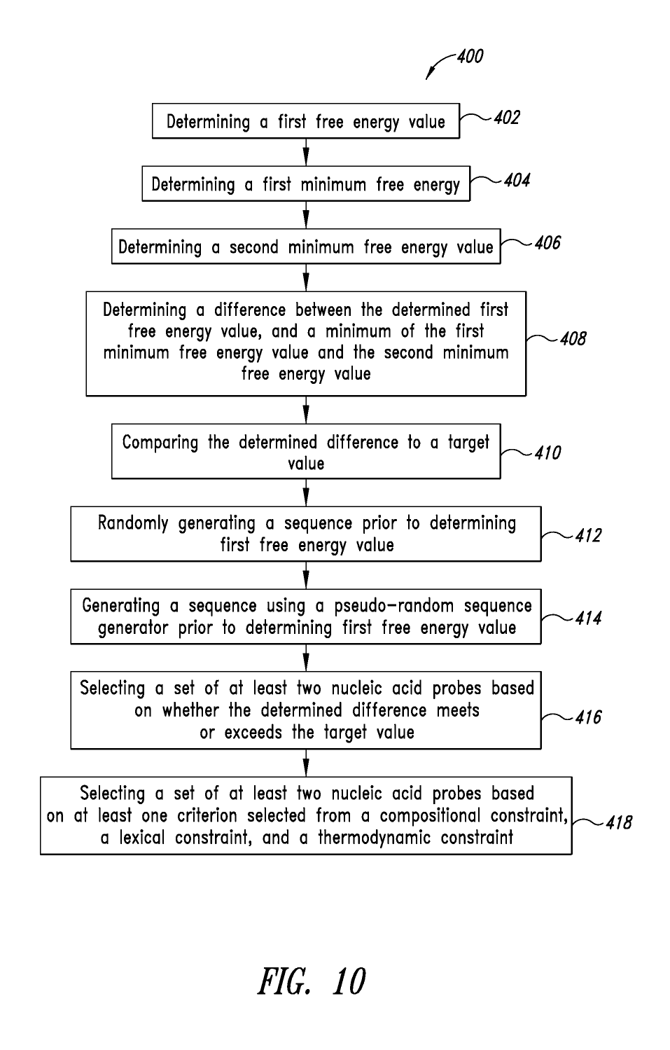

FIG. 10 shows an exemplary method 400 for analyzing nucleic acid probes using a computer system.

At 402, the method 400 includes determining a first free energy value indicative of a duplex of a first nucleic acid probe and a first target nucleic acid sequence. In some embodiments, free energy values may be determined using, for example, sequence-dependent thermodynamic parameters. In some other embodiments, free energy values may be determined using, for example, one or more nearest neighbor (n-n) modeling approaches.

In some embodiments, the free energy values may be retrieved from a data structure comprising a thermodynamic data section including thermodynamic data representative of dangling ends of two or more bases. In some embodiments, the thermodynamic data section may further include thermodynamic data representative of unpaired single strands of two or more bases adjacent to a Watson-Crick base pairing. In some embodiments, the thermodynamic data section may further include thermodynamic data representative of unpaired single strands of one or more bases adjacent to a non-Watson-Crick base pairing. In some embodiments, the thermodynamic data section may further include thermodynamic data representative of tandem base pair mismatches of two or more bases. In some embodiments, the thermodynamic data section may further include thermodynamic data representative of length-dependent terminal mismatches of nucleic acid bases. In some embodiments, the thermodynamic data section may further include thermodynamic data representative of terminal base pair mismatches.

At 404, the method 400 includes determining a first minimum free energy value indicative of a lowest free energy value associated with a formation of each of one or more duplexes formed by the first nucleic acid probe and at least a second target nucleic acid sequence. In some embodiments, determining the first free value comprises retrieving from storage a free energy contribution parameter in parallel for one or more of the comparisons of the first or the at least second nucleic acid probe base sequence, to the first or the second plurality of target bases.

At 406, the method 400 includes determining a second minimum free energy value indicative of a lowest free energy value associated with a formation of each of one or more duplexes formed by the first nucleic acid probe and at least a second nucleic acid probe.

At 408, the method 400 includes determining a difference between the determined first free energy value, and a minimum of the first minimum free energy value and the second minimum free energy value.

At 410, the method 400 includes comparing the determined difference to a target value. In some embodiments, comparing the determined difference to a target value comprises comparing the determined difference to a target minimum free energy value, a target maximum energy gap value, a target difference of free energy value, or combinations thereof.

At 412, the method 400 may further include randomly generating a sequence of the first nucleic acid probe and a sequence of the at least second nucleic acid probe prior to determining the first free energy value.

At 414, the method 400 may further include generating a sequence of the first nucleic acid probe and a sequence of the at least second nucleic acid probe using a pseudo-random sequence generator prior to determining the first free energy value.

At 416, the method 400 may further include selecting a set of at least two nucleic acid probes based on whether the determined difference meets or exceeds the target value.

At 418, the method 400 may further include selecting a set of at least two nucleic acid probes based on at least one criterion selected from a compositional constraint, a lexical constraint, and a thermodynamic constraint.

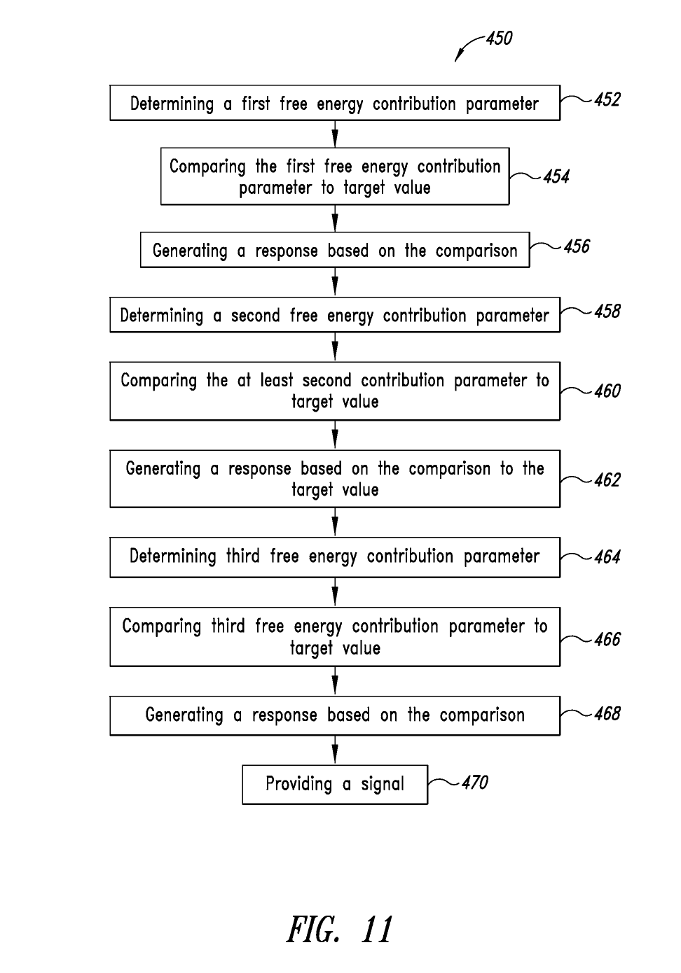

FIG. 11 shows an exemplary method 450 for determining the presence or absence of a target nucleic acid sequence in a sample using a computer system.

At 452, the method 450 includes determining a first free energy contribution parameter for a comparison of a first nucleic acid probe base sequence to a first plurality of target bases of a target sequence.

At 454, the method 450 includes comparing the first free energy contribution parameter to a target value.

At 456, the method 450 includes generating a response based on the comparison to the target value. In some embodiments, generating a response based on the comparison includes generating the response based on a comparison of the first free energy contribution parameter to a target value indicative of the presence of the target nucleic acid sequence or a closely homologous sequence. In some embodiments, generating a response based on the comparison includes having a controller 12 compare the first free energy contribution parameter to the target value, and to generate at least one of a comparison plot, comparison data, an indication of a level of gene expression, an indication of a presence or absence of one or more nucleic acid sequences, or an indication of an L-length-mer composition of a target DNA fragment based on the comparison.

At 458, the method 450 may further include determining a second free energy contribution parameter for a comparison of at least a second nucleic acid probe base sequence to the first plurality of target bases of the target sequence.

At 460, the method 450 may further include comparing the at least second contribution parameter to the target value.

At 462, the method 450 may further include generating a response based on the comparison to the target value.

At 464, the method 450 may further include determining a third free energy contribution parameter for a comparison of the first nucleic acid probe base sequence to a second plurality of target bases of a target sequence.

In some embodiments, determining the third free energy contribution parameter comprises shifting the first nucleic acid probe base sequence by at least one base in comparison to the first plurality of target bases of the target sequence to define the second plurality of target bases, and determining the third free energy contribution parameter for the comparison of the first nucleic acid probe base sequences with the second plurality of target bases.

At 466, the method 450 may further include comparing the third free energy contribution parameter to the target value.

At 468, the method 450 may further include generating a response based on the comparison to the target value.

At 470, the method 450 may further include providing a signal indicative of when the first free energy parameter is less than a target threshold amount.

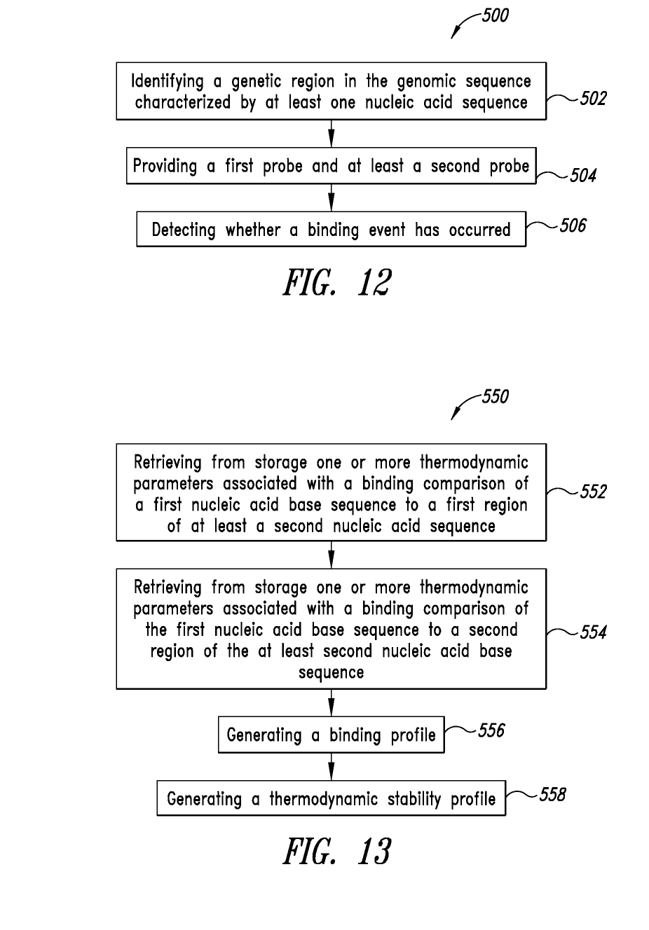

FIG. 12 shows an exemplary method 500 for analyzing a genomic sequence.

At 502, the method 500 includes identifying a genetic region in the genomic sequence characterized by at least one nucleic acid sequence.

At 504, the method 500 includes providing a first probe and at least a second probe, the first and the at least second probes may be provided based on a free energy gap characteristic indicative of a binding affinity for the at least one nucleic acid sequence.

At 506, the method 500 includes detecting whether a binding event between the first and the at least second probes and the at least one nucleic acid sequence has occurred.

FIG. 13 shows an exemplary method 550 for determining the thermodynamic characteristics of nucleic acid sequences.

In some embodiments, at least one computer readable storage medium stores instructions that, when executed on a computer, execute the method 550 for determining the thermodynamic characteristics of nucleic acid sequences.

At 552, the method 550 includes retrieving from storage one or more thermodynamic parameters associated with a binding comparison of a first nucleic acid base sequence to a first region of at least a second nucleic acid base sequence. In some embodiments, retrieving from storage one or more thermodynamic parameters comprises retrieving from storage at least one value indicative of a nearest-neighbor free energy parameter, a nearest-neighbor enthalpy parameter, or a nearest-neighbor entropy parameter.

At 554, the method 550 may further include retrieving from storage one or more thermodynamic parameters associated with a binding comparison of the first nucleic acid base sequence to a second region of the at least second nucleic acid base sequence, the second region different from the first region by at least one nucleic acid base position along a nucleic acid sequence of the second nucleic acid base sequence.

The one or more thermodynamic parameters may comprise at least one of a dangling end of two or more bases thermodynamic parameter, an unpaired single strand of two or more bases adjacent to a Watson-Crick base pairing thermodynamic parameter, a tandem base pair mismatch of two or more bases thermodynamic parameter, a length-dependent terminal mismatch of nucleic acid base thermodynamic parameter, and a terminal base pair mismatch thermodynamic parameter.

At 556, the method 550 may further include generating a binding profile for the first nucleic acid base sequence based on the comparison of the first nucleic acid base sequence to the first region, or the comparison of the first nucleic acid base sequence to the second region.

At 558, the method 550 may further include generating a thermodynamic stability profile for the first nucleic acid base sequence based on the comparison of the first nucleic acid base sequence to the first region, or the comparison of the first nucleic acid base sequence to the second region. Referring to FIGS. 2B and 2C, as previously noted, the thermodynamic stability of two stranded complexes 100, in some embodiments, may be determined from the sum 106, 122 of n-n interactions over all n-n doublets in the duplex.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The teachings provided herein of the various embodiments can be applied to systems, devices, and methods for analyzing biological samples, analyzing biological molecules (e.g., oligonucleotides, peptides, proteins, or the like), nucleic acid probes, evaluating thermodynamic properties of nucleic acid sequences, or the like, not necessarily the exemplary systems, devices, and methods for analyzing biological samples, analyzing biological molecules (e.g., oligonucleotides, peptides, proteins, or the like), nucleic acid probes, evaluating thermodynamic properties of nucleic acid sequences, or the like generally described above.

For instance, the foregoing detailed description has set forth various embodiments of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one embodiment, the present subject matter may be implemented via Application Specific Integrated Circuits (ASICs). However, those skilled in the art will recognize that the embodiments disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

In addition, those skilled in the art will appreciate that the mechanisms taught herein are capable of being distributed as a program product in a variety of forms, and that an illustrative embodiment applies equally regardless of the particular type of signal bearing media used to actually carry out the distribution. Examples of signal bearing media include, but are not limited to, the following: recordable type media such as floppy disks, hard disk drives, CD ROMs, digital tape, and computer memory; and transmission type media such as digital and analog communication links using TDM or IP based communication links (e.g., packet links).

The various embodiments described above can be combined to provide further embodiments. To the extent that they are not inconsistent with the specific teachings and definitions herein, all of the U.S. patents, U.S. patent application publications, U.S. patent applications, foreign patents, foreign patent applications and non-patent publications referred to in this specification and/or listed in the Application Data Sheet, including but not limited to U.S. Provisional Patent Application No. 60/884,161 filed Jan. 9, 2007; and U.S. Provisional Patent Application No. 60/947,597 filed Jul. 2, 2007, are incorporated herein by reference, in their entirety. Aspects of the embodiments can be modified, if necessary, to employ, for example, systems, circuits, and concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A data processing system for analyzing a biological sample, comprising:
a computer-readable memory medium comprising thermodynamic data configured as a data structure for use in analyzing biological samples, the data structure comprising: a thermodynamic data section having:
thermodynamic data representative of dangling ends of two or more bases,
thermodynamic data representative of unpaired single strands of two or more bases adjacent to a Watson-Crick base pairing,
thermodynamic data representative of unpaired single strands of one or more bases adjacent to a non-Watson-Crick base pairing,
thermodynamic data representative of tandem base pair mismatches of two or more bases,
thermodynamic data representative of length-dependent terminal mismatches of nucleic acid base, and
thermodynamic data representative of terminal base pair mismatches, or combinations thereof; and
a controller configured to compare an input associated with the biological sample to the thermodynamic data, and to generate a response based on the comparison;
wherein the input associated with the biological sample comprises at least of one of an output generated from a detected image of the biological sampled applied to an array, gene expression data, nucleic acid sequence data, an n-dimensional expression profile vector of the biological sample, a genome of an organism, or combinations thereof.

2. The system of claim 1, wherein thermodynamic data section further comprises:
thermodynamic data representative of dangling ends of a single nucleic acid base,
thermodynamic data representative of Watson-Crick base pairings,
thermodynamic data representative of single base pairings of mismatched doublets,
thermodynamic data representative of initial binding processes, or combinations thereof.

3. The system of claim 1 wherein the thermodynamic data comprises nearest-neighbor free energy values, nearest-neighbor enthalpy values, or nearest-neighbor entropy values, or combinations thereof.

4. The system of claim 1 wherein the thermodynamic data comprises binding affinity data indicative of a nucleic acid base sequence binding affinity to a target, and stability data indicative of a thermodynamic stability of a nucleic acid base sequence bound to the target, or combinations thereof.

5. The system of claim 1 wherein the thermodynamic data comprises salt concentration-dependent thermodynamic data, buffer concentration-dependent thermodynamic data, sample concentration-dependent thermodynamic data, temperature-dependent thermodynamic data, or combinations thereof.

6. The system of claim 1 wherein the controller is configured to compare the input associated with the biological sample to the thermodynamic data, and to generate at least one of a comparison plot, comparison data, an indication of a level of gene expression, an indication of a presence or absence of one or more nucleic acid sequences, or an indication of an L-length-mer composition of a target DNA fragment based on the comparison.

7. The system of claim 1 wherein the computer-readable memory medium comprises one or more field-programmable gate arrays comprising one or more look-up tables.

8. A method in a computer system for analyzing nucleic acid probes, comprising:
determining a first free energy value indicative of a duplex of a first nucleic acid probe and a first target nucleic acid sequence;
determining a first minimum free energy value indicative of a lowest free energy value associated with a formation of each of one or more duplexes formed by the first nucleic acid probe and at least a second target nucleic acid sequence;
determining a second minimum free energy value indicative of a lowest free energy value associated with a formation of each of one or more duplexes formed by the first nucleic acid probe and at least a second nucleic acid probe;
determining a difference between the determined first free energy value, and a minimum of the first minimum free energy value and the second minimum free energy value; and
comparing the determined difference to a target value.

9. The method of claim 8, further comprising:
randomly generating a sequence of the first nucleic acid probe and a sequence of the at least second nucleic acid probe prior to determining the first free energy value.

10. The method of claim 8, further comprising:
generating a sequence of the first nucleic acid probe and a sequence of the at least second nucleic acid probe using a pseudo-random sequence generator prior to determining the first free energy value.

11. The method of claim 8 wherein comparing the determined difference to a target value comprises comparing the determined difference to a target minimum free energy value, a target maximum energy gap value, a target difference of free energy value, or combinations thereof.

12. The method of claim 8, further comprising:
selecting a set of at least two nucleic acid probes based on whether the determined difference meets or exceeds the target value.

13. The method of claim 8, further comprising:
selecting a set of at least two nucleic acid probes based on at least one criterion selected from a compositional constraint, a lexical constraint, and a thermodynamic constraint.

14. A method in a computer system for determining the presence or absence of a target nucleic acid sequence in a sample, comprising:
determining a first free energy contribution parameter for a comparison of a first nucleic acid probe base sequence to a first plurality of target bases of a target sequence;
comparing the first free energy contribution parameter to a target value; and
generating a response based on the comparison to the target value.

15. The method of claim 14 wherein generating a response based on the comparison includes generating the response based on a comparison of the first free energy contribution parameter to a target value indicative of the presence of the target nucleic acid sequence or a closely homologous sequence.

16. The method of claim 14 further comprising:
determining a second free energy contribution parameter for a comparison of at least a second nucleic acid probe base sequence to the first plurality of target bases of the target sequence;
comparing the at least second contribution parameter to the target value; and
generating a response based on the comparison to the target value.

17. The method of claim 14, further comprising:
determining a third free energy contribution parameter for a comparison of the first nucleic acid probe base sequence to a second plurality of target bases of a target sequence;
comparing the third free energy contribution parameter to the target value; and
generating a response based on the comparison to the target value.

18. The method of claim 17 wherein determining the third free energy contribution parameter comprises shifting the first nucleic acid probe base sequence by at least one base in comparison to the first plurality of target bases of the target sequence to define the second plurality of target bases, and determining the third free energy contribution parameter for the comparison of the first nucleic acid probe base sequences with the second plurality of target bases.

19. The method of claim 17 wherein determining a first free energy contribution parameter comprises retrieving from storage the free energy contribution parameter in parallel for one or more of the comparisons of the first or the at least second nucleic acid probe base sequence, to the first or the second plurality of target bases.

20. The method of claim 14, further comprising:
providing a signal indicative of when the first free energy parameter is less than a target threshold amount.

21. A computer-readable memory medium containing instructions for controlling a computer processor to store in a data repository a data structure representing a comparison of a first plurality of nucleic acids with at least a second plurality of nucleic acids, by:
determining one or more duplex interactions formed between the first plurality of nucleic acids and the at least second plurality of nucleic acids, the duplex interactions selected from dangling ends of two or more bases, unpaired single strands of two or more bases adjacent to a Watson-Crick base pairing, unpaired single strands of one or more bases adjacent to a non-Watson-Crick base pairing, tandem base pair mismatches of two or more bases, length-dependent terminal mismatches of nucleic acid base, terminal base pair mismatches, Watson-Crick base pairings, single base pairings of mismatched doublets, initial binding processes, and combinations thereof; and
storing sets of thermodynamic values indicative of each of the one or more duplex interactions formed between the first plurality of nucleic acids and the at least second plurality of nucleic acids.

22. At least one computer readable storage medium comprising instructions that, when executed on a computer, execute a method for determining the thermodynamic characteristics of nucleic acid sequences, comprising:
retrieving from storage one or more thermodynamic parameters associated with a binding comparison of a first nucleic acid base sequence to a first region of at least a second nucleic acid base sequence; and
retrieving from storage one or more thermodynamic parameters associated with a binding comparison of the first nucleic acid base sequence to a second region of the at least second nucleic acid base sequence, the second region different from the first region by at least one nucleic acid base position along a nucleic acid sequence of the second nucleic acid base sequence;
wherein the one or more thermodynamic parameters comprise at least one of a dangling end of two or more bases thermodynamic parameter, an unpaired single strand of two or more bases adjacent to a Watson-Crick base pairing thermodynamic parameter, a tandem base pair mismatch of two or more bases thermodynamic parameter, a length-dependent terminal mismatch of nucleic acid base thermodynamic parameter, and a terminal base pair mismatch thermodynamic parameter.

23. The computer readable storage medium of claim 22, further comprising:
generating a binding profile for the first nucleic acid base sequence based on the comparison of the first nucleic acid base sequence to the first region, or the comparison of the first nucleic acid base sequence to the second region.

24. The computer readable storage medium of claim 22, further comprising:
generating a thermodynamic stability profile for the first nucleic acid base sequence based on the comparison of the first nucleic acid base sequence to the first region, or the comparison of the first nucleic acid base sequence to the second region.

25. The computer readable storage medium of claim 22 wherein retrieving from storage one or more thermodynamic parameters comprises retrieving from storage at least one value indicative of a nearest-neighbor free energy parameter, a nearest-neighbor enthalpy parameter, or a nearest-neighbor entropy parameter.

26. A computing device for evaluating thermodynamic properties of a nucleic acid probe and a target nucleic acid sequence, comprising:
an integrated circuit having a plurality of logic components;
an input device coupled to the integrated circuit, the input device operable to provide data indicative of one or more thermodynamic characteristics of a comparison of individual base pair binding events associated with a nucleic acid probe and at least a first region of a nucleic acid sequence; and
a processor coupled to the integrated circuit, the processor operable to analyze an output of one or more of the plurality of logic components and to determine a thermodynamic free energy of the comparison of the individual base pair binding events associated with the nucleic acid probe and the at least first region of the nucleic acid sequence.

27. The device of claim 26 wherein the integrated circuit is a field programmable gate array having a plurality of programmable logic components.

28. The device of claim 26 wherein the integrated circuit is an application specific integrated circuit having a plurality of predefined logic components.

29. A method for analyzing a genomic sequence, comprising:
identifying a genetic region in the genomic sequence characterized by at least one nucleic acid sequence;
providing a first probe and at least a second probe, the first and the at least second probes provided based on a free energy gap characteristic indicative of a binding affinity for the at least one nucleic acid sequence; and
detecting whether a binding event between the first and the at least second probes and the at least one nucleic acid sequence has occurred.

30. A computer system for analyzing nucleic acid probes, comprising:
a computer-readable memory medium comprising thermodynamic data associated with at least one of a first nucleic acid sequence and a second nucleic acid sequence, the thermodynamic data configured as a data structure; and
a shift register structure comprising:
a first set of shift registers having a first plurality of shift registers interconnected in series, at least one of the first plurality of registers configured to receive a clock signal having a shift frequency, the first set of shift registers configured to shift thermodynamic data associated with the first nucleic acid sequence loaded into at least one shift register in the first set of shift registers to a next one of a shift register in the first set of shift registers according to the shift frequency; and
a second set of shift registers having a second plurality of shift registers interconnected in series, the second set of shift registers having one or more shift register loaded with thermodynamic data associated with the second nucleic acid sequence;
wherein the shift register structure is configure to generate a comparison of thermodynamic data associated with the first nucleic acid sequence loaded in one or more shift register in the first set of shift registers and thermodynamic data associated with the second nucleic acid sequence loaded in one or more shift register in the second set of shift registers.
